(19) United States
(12) Patent Application Publication
(10) Pub. No.: US 2024/0390851 A1
(43) Pub. Date: Nov. 28, 2024

(54) PHOTOCATALYTIC AEROBIC OXIDATION OF YPERITE OR AN ANALOG THEREOF

(71) Applicant: COMMISSARIAT A L'ENERGIE ATOMIQUE ET AUX ENERGIES ALTERNATIVES, PARIS (FR)

(72) Inventors: Eric DORIS, GIF-SUR-YVETTE (FR); Edmond GRAVEL, GIF-SUR-YVETTE (FR); Emmanuel OHEIX, MULHOUSE (FR)

(21) Appl. No.: 18/692,624

(22) PCT Filed: Nov. 4, 2022
(86) PCT No.: PCT/EP2022/080872

§ 371 Date: Mar. 15, 2024

(30) Foreign Application Priority Data
Nov. 5, 2021 (EP) 21306560.0

(51) Int. Cl. B01D 53/86; B01D 53/00; B01D 53/88; B01D 69/14; B01J 31/02; B01J 35/39; C07C 315/02

(52) U.S. Cl. CPC B01D 53/8606; B01D 53/007; B01D 53/8662; B01D 53/885; B01D 69/145; B01J 31/0235; B01J 35/39; C07C 315/02; B01D 2251/11; B01D 2255/70; B01D 2257/2064; B01D 2257/306; B01D 2258/06; B01D 2259/802; B01J 2231/70

(57) ABSTRACT

The present invention relates to method for converting a sulfide of the following formula (I), such as yperite: R¹—S—R² (I) wherein R¹ and R², identical or different, are a (C₁-C₃)alkyl, (C₂-C₃)alkenyl, aryl, aryl-(C₁-C₃)alkyl, or aryl-(C₂-C₃)alkenyl group, said group being optionally substituted by one or several groups selected from a halogen atom, OR³, and NR⁴R⁵, and R³, R⁴, and R⁵ are, independently of one another, H or a (C₁-C₃)alkyl; into a sulfoxide of the following formula (II): R¹—SO—R² (II) wherein R¹ and R², are as defined above, wherein said method comprises oxidizing the sulfide of formula (I) in the presence of a catalyst. under an atmosphere comprising dioxygen, and under white or blue light irradiation, wherein the catalyst has the following formula (I): wherein Ar¹, Ar², Ar³, and Ar⁴ are, independently of one another, an aryl group optionally substituted by one or several groups selected from halogen, a (C₁-C₃)alkyl, OR⁶, and NR⁷R⁸, and R⁶, R⁷, and R⁸ are, independently of one another, H or a (C₁-C₃)alkyl. The present invention relates also to an air-filtering device comprising a catalyst of formula (I).

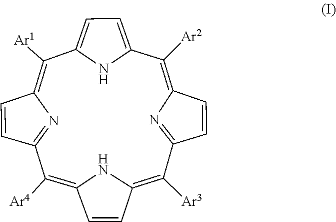

### TECHNICAL FIELD

The present invention relates to a method for the photocatalytic aerobic oxidation of yperite or an analog thereof into the corresponding sulfoxide, which is a non-toxic substance.

### BACKGROUND

Yperite (also called mustard gas, sulfur mustard or 1-chloro-2-[(2-chloroethyl)sulfanyl]ethane) is a chemical warfare agent which was mostly used during world war conflicts in the form of dispersed aerosols. Nowadays, its production and storage are prohibited by international treaties, but its revival by ill-intentioned individuals cannot be excluded, considering the simplicity of its synthesis. There is thus continued interest in the development of suitable protective equipment, air-filtering devices, and degradation methods.

The chemical neutralization of yperite (A) (i.e. the degradation or conversion of yperite into non-toxic substance(s)) can proceed via either hydrolysis, β-elimination, or sulfur oxidation (references [1]-[4]). The latter option is particularly appealing but requires the process to be selective toward the harmless sulfoxide derivative (B), rather than the toxic sulfone (C) (reference [5]).

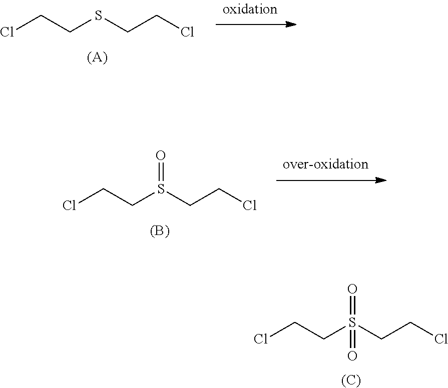

Besides selectivity issues, an “ideal” transformation should also involve non-polluting reagents and generate no side-products, making dioxygen particularly attractive as the oxidant source in case of chemical neutralization by sulfur oxidation.

Recently, a selective photocatalytic aerobic oxidation of yperite or a simulant thereof into sulfoxide has been developed. This reaction needs to be performed in the presence of an appropriate photocatalyst (a porphyrin complex), in a solvent medium, i.e. in a liquid phase, and under dioxygen (see references [6]-[9]). This reaction is generally completed within a few minutes under pure oxygen atmosphere, but require extended reaction times under air. Moreover, since the main risk of exposure to yperite is from airborne aerosols, it is important to be able to degrade yperite in an aerosol or gas phase.

Only a few studies report the decomposition of sulfur mustard in a gas phase (reference [10]-[13]). This decomposition is performed by photochemical processes involving the mineralization of gaseous yperite or a simulant thereof on a semi-conductor material (TiO₂), and under UV irradiation, either in a flow circulating reactor (reference [11]-[12]) or a quartz cell (reference [13]). However, in addition to the fact that these processes are rather complex to implement, they usually involve over-stoichiometric amounts of the photocatalyst, display low selectivity and lead to a mixture of degradation products, the toxicity of which is not known, and often result in the rapid surface deactivation of the catalyst.

There is thus still a need for a method allowing the degradation of yperite or an analog thereof into non-toxic substance(s) in an aerosol or gas phase, such as by oxidation of yperite or an analog thereof into sulfoxide.

### SUMMARY OF THE INVENTION

The present invention allows overcoming the problems of the prior art by providing a method for the degradation of yperite or an analog thereof by photocatalytic aerobic oxidation that:
can be performed in an aerosol or gas phase (but also in a liquid phase);
is very simple to implement since it can be performed in a simple round-bottom flask made of glass at the laboratory scale;
can be performed under an air atmosphere, i.e. using dioxygen present in the air as oxidant;
needs only white or blue light, including sunlight, in particular white light, and low amounts of catalyst (e.g. 0.1 mol %) to activate the oxidation reaction;
involves a catalyst that can be recycled, i.e. recovered and reused for several cycles (e.g. 10 cycles).
   Moreover, such a process has a good selectivity and leads mainly to the formation of the sulfoxide as degradation product.

The present invention thus relates to a method for converting a sulfide of the following formula (I) (i.e. yperite or an analog thereof):

R¹—S—R² (I)
wherein R¹ and R², identical or different, are a (C₁-C₃)alkyl, (C₂-C₃)alkenyl, aryl, aryl-(C₁-C₃)alkyl, or aryl-(C₂-C₃)alkenyl group, said group being optionally substituted by one or several groups selected from a halogen atom (e.g. Cl), OR³ (e.g. OH), and NR⁴R⁵, and
R³, R⁴, and R⁵ are, independently of one another, H or a (C₁-C₃)alkyl, into a sulfoxide of the following formula (II):

R¹—SO—R² (II)
wherein R¹ and R², are as defined above,
wherein said method comprises oxidizing the sulfide of formula (I) in the presence of a catalyst, under an atmosphere comprising dioxygen, and under white or blue light irradiation, in particular white light irradiation,
wherein the catalyst has the following formula (I):

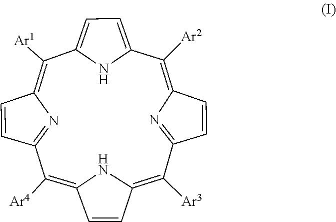

wherein Ar¹, Ar², Ar³, and Ar⁴ are, independently of one another, an aryl group optionally substituted by one or several, in particular 1, 2, or 3 groups selected from halogen, a (C₁-C₃) alkyl, OR⁶, and NR⁷R⁸, and
R⁶, R⁷, and R⁸ are, independently of one another, H or a (C₁-C₃)alkyl.

The present also relates to an air-filtering device comprising a catalyst of formula (I) as defined above.

### DEFINITIONS

The term “(C₁-C₃) alkyl”, as used in the present invention, refers to a straight or branched monovalent saturated hydrocarbon chain containing from 1 to 3 carbon atoms including, methyl, ethyl, n-propyl, or iso-propyl.

The term “(C₂-C₃) alkenyl”, as used in the present invention, refers to a straight or branched monovalent unsaturated hydrocarbon chain containing from 2 to 3 carbon atoms and comprising at least one double bond including ethenyl, and propenyl.

The term “aryl”, as used in the present invention, refers to an aromatic hydrocarbon group comprising preferably 6 to 10 carbon atoms and comprising one or more fused rings, such as, for example, a phenyl or naphthyl group. Advantageously, it will be a phenyl group.

The term “aryl-(C₁-C₃)alkyl”, as used in the present invention, refers to a (C₁-C₃)alkyl group as defined above substituted with an aryl group as defined above. In particular, it can be a benzyl group.

The term “aryl-(C₂-C₃)alkenyl”, as used in the present invention, refers to a (C₂-C₃)alkenyl group as defined above substituted with an aryl group as defined above. In particular, it can be a phenylethenyl group.

The term “halogen atom”, as used in the present invention, refers to a fluorine, bromine, chlorine or iodine atom.

The terms “catalyst” and “photocatalyst” are used interchangeably.

### DETAILED DESCRIPTION

The method according to the invention allows converting a sulfide of formula (I), i.e. yperite or an analog thereof that can be used notably as a simulant of yperite, into a sulfoxide that is a non-toxic substance. Such a method allows the neutralization of yperite, i.e. its degradation into non-toxic substance(s).

The sulfide is a sulfide of formula (I), wherein R¹ and R², identical or different, are a (C₁-C₃)alkyl, (C₂-C₃)alkenyl, aryl, aryl-(C₁-C₃)alkyl, or aryl-(C₂-C₃)alkenyl group said group being optionally substituted by one or several groups selected from a halogen atom (e.g. Cl), OR³ (e.g. OH), and NR⁴R⁵; in particular optionally substituted by one or several groups selected from a halogen atom (e.g. Cl), OH, and NH₂; more particularly optionally substituted by one group selected from a halogen atom (e.g. Cl), OH, and NH₂, preferably selected from halogen atom (e.g. Cl), and OH, especially from Cl and OH. According to a particular embodiment, at least one of R₁ and R₂ is a group substituted by one or several groups selected from a halogen atom (e.g. Cl), OR³ (e.g. OH), and NR⁴R⁵; in particular substituted by one or several groups selected from a halogen atom (e.g. Cl), OH, and NH₂; more particularly substituted by one group selected from a halogen atom (e.g. Cl), OH, and NH₂, preferably selected from halogen atom (e.g. Cl), and OH, especially from Cl and OH.

The sulfide can be in particular a sulfide of formula (I), wherein R¹ and R², identical or different, are a (C₁-C₃)alkyl, (C₂-C₃)alkenyl, or aryl group, said group being optionally substituted by one or several groups selected from a halogen atom (e.g. Cl), OR³ (e.g. OH), and NR⁴R⁵; in particular optionally substituted by one or several groups selected from a halogen atom (e.g. Cl), OH, and NH₂; more particularly optionally substituted by one group selected from a halogen atom (e.g. Cl), OH, and NH₂, preferably selected from halogen atom (e.g. Cl), and OH, especially from Cl and OH. According to a particular embodiment, at least one of R₁ and R₂ is a group substituted by one or several groups selected from a halogen atom (e.g. Cl), OR³ (e.g. OH), and NR⁴R⁵; in particular substituted by one or several groups selected from a halogen atom (e.g. Cl), OH, and NH₂; more particularly substituted by one group selected from a halogen atom (e.g. Cl), OH, and NH₂, preferably selected from halogen atom (e.g. Cl), and OH, especially from Cl and OH.

The sulfide can be more particularly a sulfide of formula (I), wherein R¹ and R², identical or different, are a (C₁-C₃)alkyl group, said group being optionally substituted by one or several groups selected from a halogen atom (e.g. Cl), OR³ (e.g. OH), and NR⁴R⁵; in particular optionally substituted by one or several groups selected from a halogen atom (e.g. Cl), OH, and NH₂; more particularly optionally substituted by one group selected from a halogen atom (e.g. Cl), OH, and NH₂, preferably selected from halogen atom (e.g. Cl), and OH, especially from Cl and OH. According to a particular embodiment, at least one of R₁ and R₂ is a group substituted by one or several groups selected from a halogen atom (e.g. Cl), OR³ (e.g. OH), and NR⁴R⁵; in particular substituted by one or several groups selected from a halogen atom (e.g. Cl), OH, and NH₂; more particularly substituted by one group selected from a halogen atom (e.g. Cl), OH, and NH₂, preferably selected from halogen atom (e.g. Cl), and OH, especially from Cl and OH.

According to a particular embodiment, the sulfide is yperite.

The atmosphere comprising dioxygen is preferably air.

The white light irradiation can be a sunlight irradiation. The white or blue light irradiation, in particular the white light irradiation, can be also an irradiation from a white or blue LED (light-emitting diode).

The catalyst has the formula (I) as defined above with Ar¹, Ar², Ar³, and Ar⁴ being, independently of one another, an aryl group, preferably a phenyl, optionally substituted by one or several, in particular 1, 2, or 3 groups selected from halogen, a (C₁-C₃)alkyl, OR⁶, and NR⁷R⁸, and

R⁶, R⁷, and R⁸ being, independently of one another, H or a (C₁-C₃)alkyl.

In particular, Ar¹, Ar², Ar³, and Ar⁴ are, independently of one another, an aryl group optionally substituted by one or several, in particular one (C₁-C₃)alkyl, such as a phenyl or a tolyl (i.e. a methylphenyl such as o-, m-or p-methylphenyl).

Preferably, Ar¹, Ar², Ar³, and Ar⁴ are identical, and most preferably, they are a phenyl or a tolyl.

Preferably, the catalyst is meso-tetraphenylporphyrin (TPP).

The catalyst can be used in an amount from 0.01 to 10 mol %, preferably from 0.1 to 1mol %, relatively to the molar amount of the sulfide.

According to a particular embodiment, the oxidizing step is performed in an aerosol or gas phase. In such an embodiment, the sulfide is in a gas state or in the form of an aerosol (i.e. a suspension of liquid droplets in air) when it is contacted with the catalyst. In consequence, the temperature and the pressure, preferably the temperature, may be adapted so that the sulfide is at least partially in a gas state or in the form of an aerosol. Typically, the reaction can be carried out at a temperature from 20° C. to 100° C., especially from 40° C. to 100° C.

Depending on its boiling point and its vapor pressure, the sulfide may be at least partially in a vapor state, i.e. it may be both in a vapor phase and a liquid phase which are in equilibrium. As the oxidation reaction progresses at the contact of the sulfide with the catalyst, the sulfide in the vapor phase is oxidized so that additional sulfide is vaporized to maintain the equilibrium between the gas phase and the liquid phase of the sulfide.

In such an embodiment, the catalyst is advantageously deposited on a substrate, especially a porous substrate, such as a filter paper, a filter of an air-filtering device, a piece of cloth, a cartridge of silica or alumina, etc.

According to a particular embodiment, the oxidizing step is performed in a liquid phase. In such an embodiment, the sulfide and the catalyst are present and contacted in a liquid phase. For that, the sulfide and the catalyst may by dissolved in a solvent.

The catalyst (or the substrate on which the catalyst is deposited) may be recovered at the end of the oxidizing step, and re-used in another oxidizing step. In these conditions, the catalyst may be recycled.

According to an embodiment, the method is performed in a continuous manner, preferably in an aerosol or gas phase. Such a method can be performed for example in an air-filtering device, where the air, that may contain a sulfide such as yperite, may be continuously treated/filtered.

According to another embodiment, the method is performed batchwise.

The present invention also relates to an air-filtering device comprising a catalyst of formula (I) as defined above, such as meso-tetraphenylporphyrin (TPP).

Said catalyst is preferably deposited on the filter of the air-filtering device.

In particular, the air-filtering device further comprises:
a filtering membrane impregnated with the catalyst, said membrane being made of cellulose, polytetrafluoroethylene, acrylic (co-)polymer, polyamide, polyurethane, polyimide, polypropylene, polysulfone or a mixture thereof, such as cellulose, polytetrafluoroethylene, polyurethane, polyimide, polysulfone or a mixture thereof, and/or
a ventilating means that draws the air through the filter.

In particular, the air-filtering device comprises a filter on which the catalyst is deposited and a ventilating means that draws the air through the filter.

The air-filtering device may further comprise a source of white or blue light irradiation, in particular white light irradiation. However, such a source of white or blue light irradiation is not necessary since the sunlight may be used as source of white light irradiation.

Such an air-filtering device may be used in order to perform the method according to the present invention.

The present invention is illustrated by the following non-limitative examples.

### FIGURES

FIG. 1: Experimental setup used for the photocatalytic oxidation of sulfides such as CEES in the aerosol/gas phase.

FIG. 2: Temperature monitoring inside the round-bottom flask (without sulfide/photocatalyst) for the aerosol/gas phase oxidation setup: the white LED is switched ON at t=0 min and switched OFF at t=60 min. A thermometer was inserted in the flask and the temperature was read at constant interval.

FIG. 3: ¹H-NMR spectra alignment for pure CEES, CEESO, CEESO₂ and the reaction mixture recovered after aerosol/gas-phase photocatalytic oxidation of CEES by TPP and under an air atmosphere, for 1 h.

FIG. 4: Histograms representing the percentage of conversion (dark grey) and the percentage of selectivity to sulfoxide (light grey) for successive CEES oxidation experiments in the aerosol/gas-phase re-using the same piece of paper embedded with the TPP photocatalyst (0.1 mol %). Experiments were performed in triplicate and the error bars represent the standard deviation.

FIG. 5: Photograph of the filtration device simulator used in example 3.

### EXAMPLES

### Abbreviations

BBS: di-n-butylsulfide
CEES: 2-chloroethylethylsulfide
CEPS: chloroethylphenylsulfide
EES: diethylsulfide
HEES: 2-hydroxyethylethylsulfide
GC: gas chromatography
MPS: methylphenylsulfide
NMR: nuclear magnetic resonance
TBTBS: di-tert-butylsulfide
TPP: meso-tetraphenylporphyrin
VES: vinylethylsulfide
VPS: vinylphenylsulfide

### 1. Photocatalytic Oxidation in the Aerosol/Gas Phase

### 1.1. Photocatalytic Aerobic Oxidation of CEES

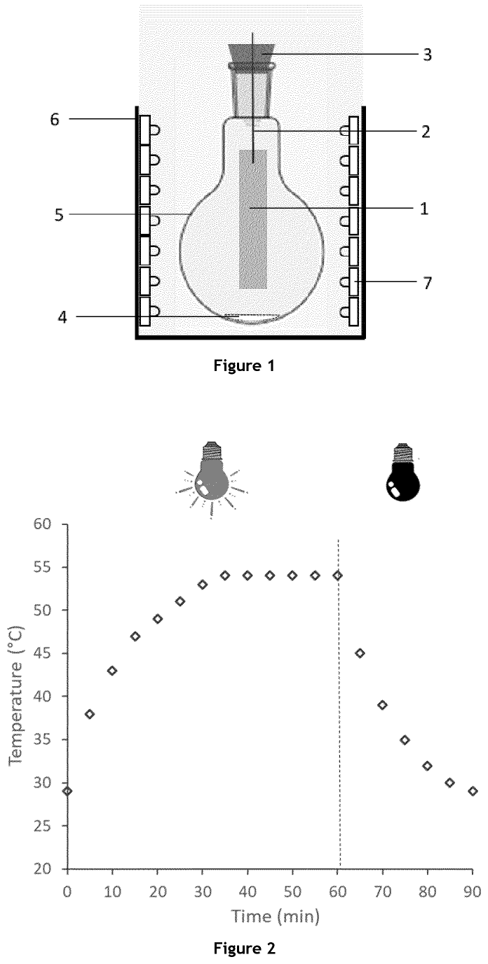

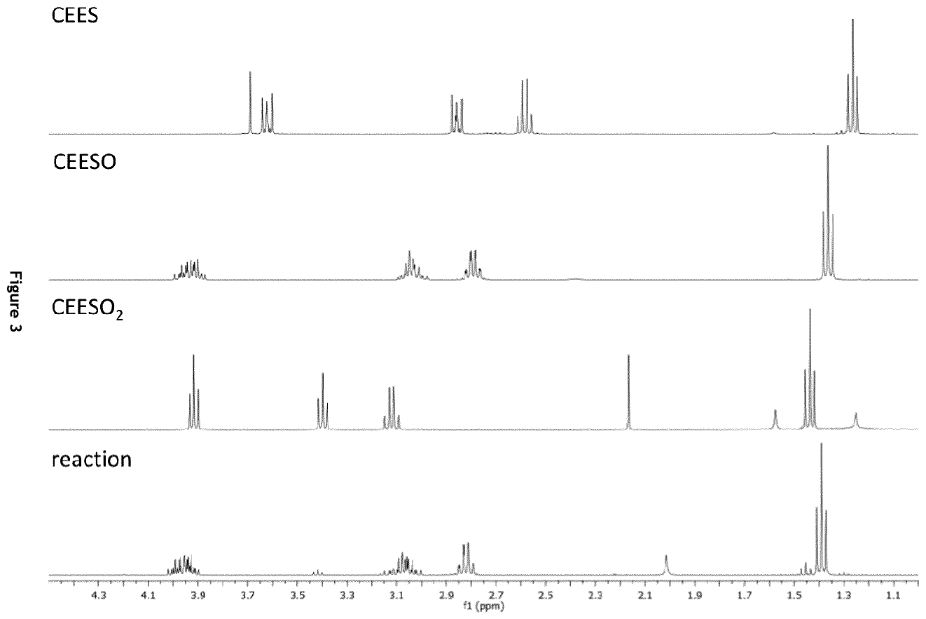

CEES was chosen as a model sulfide, i.e. as a simulant of sulfur mustard. A typical procedure is given below, the experimental setup being presented on FIG. 1. TPP (photocatalyst) (100 μL of a 2 mM solution, 0.2 μmol) in CHCl₃ is deposited on a filter paper (1×5 cm) and allowed to dry for 2 min. The filter paper embedded with the TPP photocatalyst (1) is then connected to a hook (2) attached to the inner portion of a rubber septum (3). Neat 2-chloroethylethylsulfide (4) (23.5 μL, 200 μmol) is introduced in a 25 mL round-bottom flask (5) (filled with air) which is closed with the rubber septum (connected to the paper-supported TPP). The vertical position of the paper is adjusted in order to stand 1 cm from the bottom of the round-bottom flask. The flask is positioned in a beaker (6) (7 cm diameter) fitted with white LED wires (7). The reaction is initiated by switching the LED wire ON, and it is then stopped by switching it OFF after the specified reaction time (1 h unless otherwise specified). The LEDs here serve a dual purpose, photoexcitation of TPP and gentle heating source to help vaporize CEES in the gas phase or at least convert it into an aerosol phase (FIG. 2) (if a higher temperature is needed, an additional heat source can be used). After cooling down for 5 min, the filter paper is removed and the flask and filter paper are washed with CDCl₃, transferred to a tinted NMR tube, and analyzed by ¹H-NMR and by GC. Conversion is determined from NMR analysis of the crude mixture by comparison with the NMR analysis of authentic CEES, CEESO and CEESO₂ samples (FIG. 3). For absolute quantification, a solution of dioxane (20 μL, 1 M) was added to the NMR tube to serve as an internal standard.

Experiments under N₂ and O₂ atmosphere were run by pre-purging the round-bottom flask with the suitable gas for 5 min.

The results obtained are presented in Table 1 below.

TABLE 1
Photocatalytic aerobic oxidation of CEES

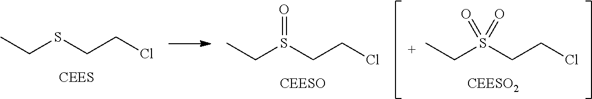

| | TPP | | | | |
|---|---|---|---|---|---|
| Entry | (mol %) | Atmosphere | Light | Conversion (%) | Selectivity (%) |
| 1 | 0.1 | air | white LED | 100 | 92 |
| 2 | 0 | air | white LED | 0 | — |
| 3 | 0.1 | air | dark | 0 | — |
| 4 | 0.01 | air | white LED | 76 | 87 |
| 5 | 1 | air | white LED | 100 | 79 |
| 6 | 0.1 | N₂ | white LED | <5 | 86 |
| 7 | 0.1 | O₂ | white LED | 100 | 38 |
| 8^{(a)} | 0.1 | air | sunlight | 100 | 84 |

^{(a)}7 h of reaction time under a mid-winter sun on a partly cloudy day

Under the conditions of the above-mentioned protocol (Entry 1), the starting CEES material was fully converted in 1 h for the most part into CEESO (expected first oxidation product, 90%), together with some minor CEESO₂ (over-oxidation product, 8%), and vinyl derivatives (B-elimination products, <2%).

Reactions run with either no TPP-catalyst (Entry 2) or in the dark (Entry 3) led to no conversion confirming that the oxidation reaction is photocatalyzed by TPP. The aerobic nature of the transformation was evidenced by running the photocatalytic reaction under an inert nitrogen atmosphere (Entry 6), which failed to provide the expected compound in satisfactory yield (<5%), while the reaction run under pure oxygen (Entry 7) mostly afforded the over-oxidized sulfone product.

Moreover, the process can be performed with higher or lower amounts of TPP (Entries 4-5) and with sunlight as irradiation source (Entry 8).

Other photocatalysts were tested under similar conditions (200 μmol CEES, 0.1 mol % photocatalyst) as reported above, except for the phthalocyanine photocatalyst which was deposited as a 1 mM suspension in CHCl₃ (due to solubility issues). No conversion was detected for phthalocyanin and (meso-tetraphenylporphyrin) iron(III), whereas a low 4% yield was obtained with (meso-tetraphenylporphyrin) zinc(II).

### 1.2. Photocatalytic Aerobic Oxidation of Various Sulfides

Other sulfides were also tested under similar conditions (200 μmol sulfide, 0.1 mol % TPP) as reported above. Reaction times were adjusted to get optimal conversion and selectivity.

The results obtained are presented in Table 2 below.

TABLE 2
Photocatalytic aerobic oxidation of different sulfides

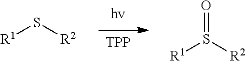

| | | | | Conversion | |
|---|---|---|---|---|---|
| Entry | Sulfide | | Time (h) | (%) | Selectivity (%) |
| 1 | CEES | 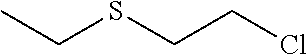 | 1 | 100 | 92 |
| 2 | HEES | 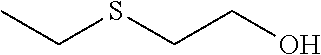 | 8 | 97 | 88 |
| 3 | BBS | 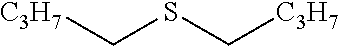 | 2 | 100 | 43 |
| 4 | TBTBS | 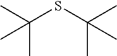 | 8 | 88 | 0 |
| 5 | EES | 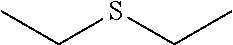 | 8 | 89 | 71 |
| 6 | VES | 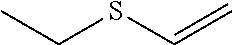 | 2 | 95 | 67 |
| 7 8^{(a)} | CEPS | 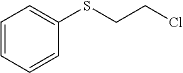 | 8 3 | 18 98 | 55 87 |
| 9 | MPS | 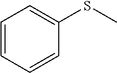 | 3 | 92 | 72 |

^{(a)}Sulfide deposited directly on the paper support.

The oxidation of HEES is complete after 8 h, which is consistent with the hydrogen bonding effect of the alcoholic group, imparting lower vapor pressure to the sulfide. Alkyl-substituted sulfides are in general more prone to over-oxidation due to the electron-donating effect of the alkyl substituents that increases the electron density of the central sulfur atom (Entries 3-4). Less electron-rich sulfides such as EES (Entry 5) and VES (Entry 6) were converted to their sulfoxide counterparts with better selectivities than those observed for butyl-substituted compounds BBS and TBTBS. Concerning aromatic sulfides, the high boiling point of CEPS (245° C.) is responsible for the mediocre reactivity/selectivity observed (Entry 7) as confirmed by the fact that the deposition of CEPS directly on the paper support affords 98% conversion with 87% selectivity (Entry 8). Besides, MPS, which has a lower boiling point (188° C.), was efficiently oxidized into the corresponding sulfoxide with a selectivity of 72% and 92% conversion (Entry 9). These results confirm that aromatic sulfides can also react satisfactorily.

### 1.3. Catalyst Recycling

Successive CEES oxidation experiments have been performed in the aerosol/gas phase according to the above-mentioned procedure by recovering and re-using the same piece of filter paper embedded with the TPP photocatalyst (0.1 mol %) with a fresh batch of CEES. The experiments were performed in triplicate.

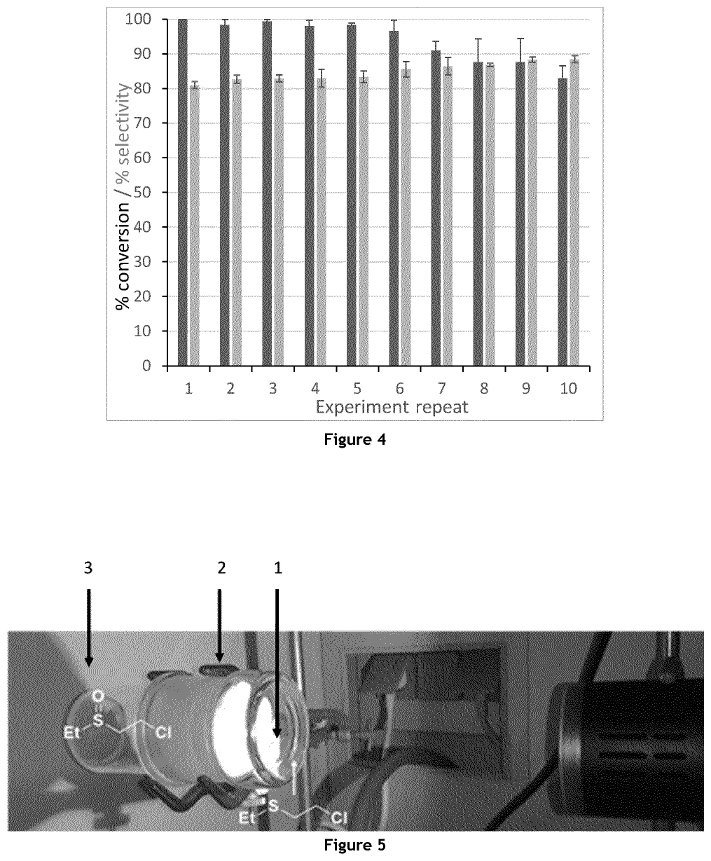

The results obtained in terms of percentage of conversion of CEES and percentage of selectivity to CEESO are presented in FIG. 4. During the first five cycles, the conversion at 1 h reaction was nearly quantitative (>98%), but gradually decreased to reach 83% after the tenth assay, although selectivity remained nearly constant (85-91%) showing that the TPP catalyst remained fully photoactive.

### 2. Photocatalytic Oxidation in the Liquid Phase

A typical procedure is given below for the oxidation of CEES.

TPP (100 μL of a 2 mM solution in CDCl₃, 0.2 μmol) is added to a 25 mL round-bottom flask containing 900 μL CD₃OD and filled with air. CEES (23.5 μL, 200 μmol) is then added and the flask is closed with a rubber septum. The flask is positioned in a beaker and illuminated with white LEDs for 1 h as done for the experimentation in an aerosol/gas phase. The product distributions are analyzed directly by ¹H-NMR. For absolute quantification, a solution of dioxane (20 μL, 1 M) was added to the NMR tube to serve as an internal standard.

Such a procedure allows the complete conversion of CEES with a selectivity to sulfoxide of 97%.

### 3. Photocatalytic Oxidation in a Filtration Device Simulator

A filtration device simulator has been used to perform a photocatalytic oxidation. A photograph of the experimental setup is presented on FIG. 5. This simulator comprises:
a first compartment (1) which is a “source” compartment containing the sulfide to be oxidized (Et-S-(CH₂)₂—Cl),
a second compartment (3) which is a collecting compartment, and.
a filtering membrane (2) (filter paper) impregnated with the catalyst (TPP) (for that, the filter paper has been impregnated with a solution of TPP in chloroform before leaving it to dry) which separates the first and second compartments (1) and (3).

The device has been exposed to blue light irradiation.

The only product identified in the collecting compartment corresponds to the oxidized form of the sulfide comprised in the “source” compartment, i.e. the sulfoxide Et-S(O)—(CH₂)₂—Cl.

### REFERENCES

[1] Smith et al., Chem. Soc. Rev., 2008, 37, 470;
[2] Jang et al., Chem. Rev., 2015, 115, PR1;
[3] Picard et al., Org. Biomol. Chem., 2019, 17, 6528;
[4] Oheix et al., Chem. Eur. J., 2021, 27, 54;
[5] Jackson, Chem. Rev., 1934, 15, 425;
[6] CN110437459;
[7] Liu et al., Angew. Chem. Int. Ed., 2015, 54, 9001;
[8] Liu et al., ACS Nano, 2015, 9 (12), 12358;
[9] Cao et al., J. Am. Chem. Soc. 2019, 141, 14505;
[10] Collins-Wildman et al., Commun. Chem., 2021, 4, 33;
[11] Vorontsov et al., Environ. Sci. Technol., 2002, 36, 5261;
[12] Vorontsov et al., J. Catalysis, 2003, 220, 414;
[13] Martyanov and Klabunde, Environ. Sci. Technol., 2003, 37, 3448.

1. A method for converting a sulfide of following formula (I):
R¹—S—R² (I)
into a sulfoxide of following formula (II):
R¹—SO—R² (II)
wherein R¹ and R², identical or different, are a (C₁-C₃)alkyl, (C₂-C₃)alkenyl, aryl, aryl-(C₁-C₃)alkyl, or aryl-(C₂-C₃)alkenyl group, which is optionally substituted by one or several groups selected from the group consisting in a halogen atom, OR³, and NR⁴R⁵, and
R³, R⁴, and R⁵ are, independently of one another, H or a (C₁-C₃)alkyl;
wherein the method comprises oxidizing the sulfide of formula (I) in the presence of a catalyst, under an atmosphere comprising dioxygen, and under white or blue light irradiation,
wherein oxidizing is performed in air used as the atmosphere comprising dioxygen, wherein the catalyst has following formula (III): 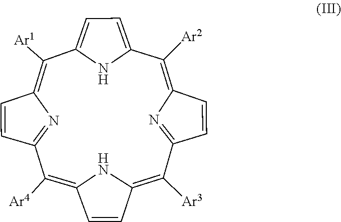
wherein Ar¹, Ar², Ar³, and Ar⁴ are, independently of one another, an aryl group optionally substituted by one or several groups selected from the group consisting in halogen, a (C₁-C₃)alkyl, OR⁶, and NR⁷R⁸, and
R⁶, R⁷, and R⁸ are, independently of one another, H or a (C₁-C₃)alkyl.

2. The method according to claim 1, wherein the sulfide is a sulfide of formula (I), wherein R¹ and R², identical or different, are a (C₁-C₃)alkyl, (C₂-C₃)alkenyl, or aryl group, which is optionally substituted by one or several groups selected from the group consisting in a halogen atom, OR³, and NR⁴R⁵.

3. The method according to claim 2, wherein the sulfide is a sulfide of formula (I), wherein R¹ and R², identical or different, are a (C₁-C₃)alkyl group optionally substituted by one group selected from the group consisting in a halogen atom, OR³, and NR⁴R⁵.

4. The method according to claim 3, wherein the sulfide is yperite.

5. The method according to claim 1, wherein Ar¹, Ar², Ar³, and Ar⁴ are, independently of one another, an aryl optionally substituted by one or several (C₁-C₃)alkyl.

6. The method according to claim 5, wherein the catalyst is meso-tetraphenylporphyrin (TPP).

7. The method according to claim 1, wherein the catalyst is used in an amount from 0.01 mol % to 10 mol % relatively to the molar amount of the sulfide.

8. The method according to claim 1, wherein the white or blue light irradiation is a sunlight irradiation or an irradiation from a white or blue light-emitting diode (LED).

9. The method according to claim 1, wherein the white or blue light irradiation is a white light irradiation.

10. The method according to claim 1, wherein the oxidizing step is performed in an aerosol or gas phase.

11. The method according to claim 10, wherein the catalyst is deposited on a substrate.

12. The method according to claim 1, wherein the catalyst is recovered at the end of the oxidizing step, and re-used in another oxidizing step.

13. The method according to claim 1, being performed in a continuous manner.

14. An air-filtering device comprising a filtering membrane impregnated with a catalyst,
wherein the filtering membrane is made of cellulose, polytetrafluoroethylene, polyurethane, polyimide, polysulfone or a mixture thereof,
and the catalyst has following formula (I): 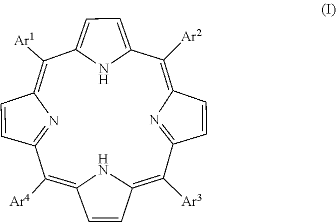
wherein Ar¹, Ar², Ar³, and Ar⁴ are, independently of one another, an aryl group optionally substituted by one or several groups selected from the group consisting in halogen, a (C₁-C₃)alkyl, OR⁶, and NR⁷R⁸, and
R⁶, R⁷, and R⁸ are, independently of one another, H or a (C₁-C₃)alkyl.

15. The air-filtering device according to claim 14, wherein the catalyst is meso-tetraphenylporphyrin (TPP).

16. The method according to claim 3, wherein R¹ and R², identical or different, are a (C₁-C₃)alkyl group optionally substituted by one group selected from the group consisting in a halogen atom, OH, and NH₂.

17. The method according to claim 3, wherein R¹ and R², identical or different, are a (C₁-C₃)alkyl group optionally substituted by one group selected from the group consisting in Cl and OH.

18. The method according to claim 1, wherein Ar¹, Ar², Ar³, and Ar⁴ are, independently of one another, a phenyl optionally substituted by one (C₁-C₃)alkyl.

19. The method according to claim 1, wherein the catalyst is used in an amount from 0.1 mol % to 1 mol % relatively to the molar amount of the sulfide.
